(19) United States
(12) Patent Application Publication
(10) Pub. No.: US 2024/0423934 A1
(43) Pub. Date: Dec. 26, 2024

(54) COMPOSITIONS IN THE FORM OF A UREA POWDER

(71) Applicant: KidneyAide, Greer, SC (US)

(72) Inventor: Barry Gorlitsky, Greer, SC (US)

(21) Appl. No.: 18/751,463

(22) Filed: Jun. 24, 2024

Related U.S. Application Data
(60) Provisional application No. 63/523,059, filed on Jun. 24, 2023.

(51) Int. Cl. A61K 31/17; A61K 45/06; A61K 47/02

(52) U.S. Cl. CPC A61K 31/17; A61K 45/06; A61K 47/02

(57) ABSTRACT

The disclosure relates to an improved composition of a mixture based on urea. The present disclosure can be broadly described as a more stable powder composition comprising an amount of urea effective for treatment of hyponatremia. The present disclosure is a urea formulation that is a sparkling Urea (may be referred to below as “UreaAide sparkling”). The usual starting dose of urea is 15 grams, twice a day. The UreaAide sparkling is a powder that can come in single use urea dose packet. In one embodiment, the UreaAide sparkling is a single use, 15 gram urea dose packet. The 15 gram urea dosage may vary. For example, the urea dosage may not be a 15 gram dose. The urea dose can be prescribed to be taken once per day or many times throughout the day.

### FIELD OF THE INVENTION

The present disclosure relates to compositions for nutrient supplementation. The dietary supplements and pharmaceutical products, as well as the methods described, may be useful in the treatment of patients with varied etiologies and chronicity of Hyponatremia.

### BACKGROUND

The kidneys carry out a multitude of excretory, metabolic, regulatory, and endocrine functions, including the maintenance of fluid and electrolyte homeostasis, the excretion of metabolic waste products, and the synthesis and degradation of various hormones. Additionally, the kidneys play a role in preserving the homeostatic composition of plasma.

Hyponatremia, characterized by plasma sodium levels below 135 mEq/L, represents a common electrolyte imbalance associated with numerous adverse physiological signs and symptoms. These symptoms encompass headache, nausea, cognitive impairment, fatigue, and balance disturbances. The condition is correlated with an increased risk of falls, fractures, osteoporosis, seizures, and mortality. Although conventional treatments for hyponatremia such as fluid restriction and oral sodium chloride tablet administration are often employed, the empirical support for these approaches remains limited. Moreover, patient adherence to these treatments tends to be low. Sodium chloride may also elevate blood pressure, cause edema and frequently fails to adequately correct sodium levels.

### SUMMARY

One aspect of the present disclosure pertains to a composition of a mixture incorporating urea. The disclosure addresses a stable powder composition comprising a certain amount of urea for the treatment of hyponatremia. Urea is a guideline-based therapy for hyponatremia. A known limitation of urea is its distaste and nausea caused by its salty/briny taste profile. This can lead to some patients discontinuing urea treatment.

In one embodiment, a urea formulation is provided as a sparkling composition, referred to as “UreaAide sparkling.” The standard initial dosage of urea is 15 grams, administered twice a day. UreaAide sparkling is supplied as a powder in single-use urea dose packets. In one embodiment, UreaAide sparkling is provided as a single-use, 15-gram urea dose packet. The 15-gram dosage is variable; for instance, the prescribed urea dosage may differ from 15 grams. The urea dose can be prescribed for once-daily or multiple-times-daily administration.

In one embodiment, UreaAide sparkling is formulated for the treatment of hyponatremia. The composition may be dispersed in water or another liquid prior to ingestion by the patient, or it may be incorporated into or mixes with food. UreaAide sparkling is provided as a powder in single-use 15-gram urea dose packets. The flavor profile of UreaAide sparkling aims to enhance taste and tolerability. In one embodiment, Agave powder is included in the formulation, which can impart a sweet flavor and has a low glycemic index. The Agave powder may also contain trace vitamins and minerals. Another embodiment includes lemon and orange flavoring to enhance taste and balance. The citrus flavor may combine with sodium bicarbonate, contributing a salty component and the sparkling attribute to improve flavor, texture, mouth-feel, and digestion. UreaAide sparkling is available in diverse flavors or flavor combinations, such as orange, mint, or blueberry-raspberry combinations. The UreaAide formulation may also include a bitter blocker. Additionally, the formulation can incorporate sucralose and stevia to enhance sweetness without adding calories.

In one embodiment, the orange flavor of the UreaAide sparkling contains Silicon Dioxide and may or may not contain Calcium Silicate. In another embodiment, the mint flavor of the UreaAide sparkling contains Calcium Silicate and may or may not contain Silicon Dioxide.

In another aspect, the disclosure relates to a powder formulation. The powder formulation includes an amount of sparkling urea effective for treatment of hyponatremia and calcium silicate. The calcium silicate has a particle size in the range of 3 to 8 microns, wherein less than 10% of the particles by weight have a diameter less than 3 microns or greater than 8 microns. In one embodiment, the urea comprises 62.50% of the formulation weight, and the urea is a sparkling UreaAide formulation. The calcium silicate may have a particle size of 8 microns.

In another aspect, the disclosure relates to a powder formulation. The powder formulation includes an amount of sparkling urea effective for treatment of hyponatremia and calcium silicate. The calcium silicate has a particle size of 8 microns.

### DETAILED DESCRIPTION

The management of hyponatremia typically involves fluid restriction and the administration of oral sodium chloride tablets. Sodium chloride tablets may be prescribed alongside fluid restriction and/or loop diuretics to elevate plasma sodium levels. However, the concurrent need for fluid restriction and/or loop diuretics may affect long-term adherence to this therapy. The use of loop diuretics in conjunction with sodium chloride and/or fluid restriction has exhibited an increased incidence of hypokalemia and acute kidney injury events compared to fluid restriction alone, and has generally not been shown to be superior.

In recent years, vasopressin receptor antagonists have been introduced as a therapeutic option for hyponatremia. However, notable safety concerns with these medications include an elevated risk of liver injury at high doses, drug interactions and the possibility of overly rapid correction of plasma sodium levels. Additionally, the high retail cost of vasopressin antagonists may limit their widespread utilization. Lastly, vasopressin receptor antagonist also have known adverse side effects that impact compliance including: excessive thirst, polyuria, nocturia and polydipsia.

Various individuals with sodium imbalances use urea, which serves as a guideline-based medical food for managing hyponatremia. Urea is synthesized by the liver as a primary end product of nitrogen metabolism, then released into the blood and excreted by the kidneys. While urea presents a more cost-effective option compared to many other hyponatremia treatments, considerations regarding its application exist, such as availability and unfavorable taste, which may deter its use as a first-line therapy for hyponatremia.

The present disclosure relates to an enhanced composition of a urea-based mixture. This disclosure can be characterized as a more stable powder composition containing an effective amount of urea for the treatment of hyponatremia. It may also pertain to treating individuals at risk of developing hyponatremia. The composition may be dispersed in water or another liquid before ingestion by the patient, or it may be incorporated into or mixed with food. Alternatively, the composition may be packaged with food until ready for use.

The present disclosure describes a urea formulation referred to as “UreaAide sparkling.” The starting dosage of urea is typically 15 grams, administered twice daily. UreaAide sparkling is a powder that can be provided in single-use urea dose packets. In one embodiment, UreaAide sparkling is a single-use, 15-gram urea dose packet. The 15-gram dose may vary. For example, the urea dosage may differ from the 15-gram dose. The urea dose can be prescribed to be taken once per day or multiple times throughout the day.

Given that urea generally has an unfavorable taste, making it difficult for patients to tolerate, the UreaAide sparkling formulation includes a specific flavor profile to enhance taste and tolerability. In one embodiment, agave powder is included in the formulation.

The agave powder, for example, may possess a sweet flavor and a low glycemic index. The agave powder may also contain trace vitamins and minerals. In one embodiment, the formulation incorporates lemon and orange flavorings to enhance taste and balance flavor. These may add a citrus sour note that, combined with sodium bicarbonate, contributes to a salty component and a sparkling element to enhance flavor, texture, mouth-feel, and digestion. UreaAide sparkling can be available in a variety of flavors or flavor combinations, such as orange, mint, or a blueberry-raspberry combination.

Blocking bitter taste perception can occur throughout the taste signal cascade. A compound can act by directly antagonizing bitter taste receptors, preventing the activation of gustducin and inhibiting taste perception. The UreaAide formulation can include a bitter blocker. A bitter blocker is a substance that inhibits or masks the perception of bitterness in food and beverages. Common bitter blockers include quinine, denatonium, and gymnemic acid. In addition to the bitter blocker, the UreaAide formulation can also include sucralose and stevia to enhance sweetness without adding calories.

UreaAide sparkling orange is a formulation low in sugar, sodium, and calories, making it suitable for individuals with diabetes and high blood pressure. In one embodiment, UreaAide sparkling orange contains only 30 calories, 3 grams of sugar, and 60 mg of sodium. It contains no fat and no cholesterol. In another embodiment, the ingredients of UreaAide sparkling orange can vary, resulting in differences in sugar, calories, fat, or other components. Although the formulation is named UreaAide sparkling orange, the flavor does not have to be orange. In one embodiment, the UreaAide sparkling formulation can be any flavor or a combination of flavors. Additionally, in one embodiment, the formulation does not have to be a sparkling formulation.

UreaAide sparkling orange can utilize a specific 8-micron-sized calcium silicate powder to enhance flow through a sachet machine, reduce clumping, and ensure consistent fill of sachets. A sachet can be a small porous bag or packet.

The particle size distribution of the calcium silicate is significant such that amounts of calcium silicate above the upper limit microns and below the lower limit are undesirable. For example, it is desirable that the particle size distribution of the calcium silicate should be adapted such that amounts below 3.0 microns and above 8.0 microns are avoided. It is preferred that the calcium silicate have a mean particle diameter within the range of 3.0 and 8.0 microns, with less than 10% of the particles by weight being below 3.0 microns or above 8.0 microns in diameter. In one embodiment, the threshold of 10% of particles by weight being outside the target particle diameter range is acceptable. Preferably, even lower amounts of off-target particles are desirable, such as 8%, 6%, 4%, or even 1%.

Table 1 is an embodiment of a specification sheet of the urea.

TABLE 1

| | | Specification | |
|---|---|---|---|
| Test | Method | MIN | MAX |
| Description | Visual | White pearls | |
| Odor | Olfactory | Faint Ammonia Odor | |
| Assay, % | USP | 99.0 | 100.5 |
| Identification | USP | To Meet Requirements | |
| Infrared Absorption | <197K> | Corresponds to Standard | |
| Retention Time of Major Peak | <621> | Corresponds to Standard | |
| Meeting Range, deg. C | <741> | 132 | 135 |
| Residue on Ignition, % | <281> | | 0.1 |
| Alcohol-insoluble matter, % | USP | | 0.04 |
| Chloride, % | <221> | | 0.007 |
| Sulfate | <221> | | 0.010 |
| Heavy Metals, ppm | <231> | | 20 |

| | | |
|---|---|---|
| Residual Solvents | <467> | To Meet Requirements |

As shown in Table 1, in one embodiment, the assay percentage is between 99% and 100.5%, the alcohol-insoluble matter is 0.04%, the chloride is 0.007%, the sulfate is 0.010%, and the heavy metals is 20 ppm or parts per million (the number of units of mass of a contaminant per million units of total mass). These are examples, the percentages and amounts may vary.

Table 2 is an embodiment of a final formulation specification of UreaAide sparkling.

TABLE 2
Product Name: UreaAide Orange flavor, 24 grams.

| Ingredient Description | mg per serving | Ingredient % |
|---|---|---|
| Urea Pellets | 15000 | 63.16% |
| Sodium Dioxide | 250 | 1.05% |
| Agave Powder | 4525 | 19.05% |
| Lemon Juice Powder | 800 | 3.37% |
| Masking Flavor A19- | 200 | 0.84% |
| 004379# | | |
| Masking Flavor A19- | 500 | 2.11% |
| 005813** | | |
| Citric Acid | 850 | 3.58% |
| Baking Soda | 300 | 1.26% |
| Orange Flavor WONF SD | 550 | 2.32% |
| A19-003935\hat{ }\hat{ } | | |
| Orange Flavor PWD A19- | 550 | 2.32% |
| 003634″″ | | |
| Stevia 95% Extract | 100 | 0.42% |
| Sucralose | 125 | 0.53% |
| | 23750 | 100.00% |

Link to all the certifications and documents:
Urea Aide Ingredient CofA's and Specs-Google Drive
#Maltodextrin + Natural Flavors
**Potassium Bitartrate 80-90%
Maltodextrin10-15%
Natural Flavors <2%
\hat{ }\hat{ }Maltodextrin, Natural Flavors, Gum Arabica
″″Arabic Gun, Natural Flavors

As shown in Table 2, the ingredient description and percentages are displayed. Urea, granule form, USP makes up 63.16% of the formulation, Prinova makes up 1.05% of the formulation, agave powder makes up 19.05% of the formulation, lemon powder makes up 3.37% of the formulation, Bitter Blocker makes up 2.95% of the formulation, citric acid makes up 3.58% of the formulation, baking soda makes up 1.26% of the formulation, orange flavor WONF (with other natural flavors) makes up 2.32% of the formulation, orange flavor PWD (powdered) makes up 2.32% of the formulation, stevia 95% extract makes up 0.42% of the formulation, sucralose makes up 0.53% of the formulation.

Table 3 is another embodiment of a final formulation specification of UreaAide sparkling.

TABLE 3
Product Name: UreaAide Blueberry Raspberry flavor

| Ingredient Description | Percentages |
|---|---|
| Urea, USP | 62.5% |
| Agave powder | 20.85% |
| Lemon powder | 4.17% |
| Sour enhancing flavor a19-003783 | 0.42% |
| Bitterblocker 655771 | 2.08% |
| Citric acid | 3.44% |
| Baking soda | 1.25% |
| Blueberry flavor | 2.08% |
| Mixed berry | 2.29% |
| Stevia 95% Extract | 0.42% |
| Calcium Silicate | 0.5% |

As shown in Table 3, the ingredient description and percentages in this embodiment are different than in Table 3. Agave powder makes up 20.85% of the formulation, lemon powder makes up 4.170% of the formulation, sour enhancing flavor makes up 0.420% of the formulation, Bitter Blocker makes up 2.08% of the formulation, citric acid makes up 3.44% of the formulation, baking soda makes up 1.25% of the formulation, blueberry flavor makes up 2.08% of the formulation, mixed berry makes up 2.290% of the formulation, stevia 9500 extract makes up 0.42% of the formulation, and calcium silicate makes up 0.50% of the formulation.

Table 4 is an embodiment of nutrition facts of UreaAide sparkling.

TABLE 4
Product Name: UreaAide Orange flavor-Nutrition Facts

| Serving size | | (9 g) |
|---|---|---|
| Amount per serving Calories | | 30 |
| % daily value | | |
| Total Fat | 0 g | 0% |
| Saturated Fat | 0 g | 0% |
| Trans Fat | 0 g | 0% |
| Cholesterol | 0 g | 0% |
| Sodium | 60 mg | 3% |
| Total Carbohydrates | 7 g | 3% |
| Dietary Fiber | 0 g | 0% |
| Total Sugars | 3 g | 0% |
| Includes 3 g Added Sugars | | 6% |
| Protein | 0 g | |
| Vitamin D | 0 mcg | 0% |
| Calcium | 4 mg | 0% |
| Iron | 0 mg | 0% |
| Potassium | 5 mg | 0% |

The UreaAide sparkling orange has ingredients of agave powder, natural flavors, citric acid, lemon powder, sodium bicarbonate, sucralose, silicon dioxide and stevia extract. The UreaAide sparkling orange has 0 grams of fat, 0 mg. of cholesterol, 60 mg. of sodium, 7 grams of total carbohydrates, 0 grams of dietary fiber, 3 grams of total sugars, and 3 grams of added sugars. In one embodiment, the UreaAide sparkling orange has 0 mcg of vitamin D, 4 mg. of Calcium, 0 mg. of Iron, and 5 mg. of Potassium. In one embodiment, the UreaAide sparkling formulation has a net weight of 24 g and is 15 g of Urea per packet.

TABLE 5
is another embodiment of a UreaAide Sparkling
Orange flavor composition:
Formula: FV.001.

| Ingredient Description | mg per serving | Ingredient % |
|---|---|---|
| Urea Pellets | 15000 | 62.50% |
| Silicon Dioxide | 250 | 1.04% |
| Agave Powder | 4775 | 19.90% |
| Lemon Juice Powder | 800 | 3.33% |
| Maltodextrin + Natural | 200 | 0.83% |
| Flavors | | |
| Potassium Bitartrate | 500 | 2.08% |
| (80-90%) + | | |
| Maltodextrin | | |
| (10-15%) + | | |
| Natural Flavors <2% | | |
| Citric Acid | 850 | 3.54% |
| Baking Soda | 300 | 1.25% |
| Maltodextrin, Natural | 550 | 2.29% |
| Flavors, Gum Arabica | | |
| Arabic Gum, Natural | 550 | 2.29% |
| Flavors | | |
| Stevia 95% Extract | 100 | 0.42% |
| Sucralose | 125 | 0.52% |
| | 24000 | 100.00% |

In one embodiment, the Urea granules are mill grinded to a powder. In one embodiment, immediately thereafter, the flavor ingredients are blended together with the ground Urea granules. Then, the 8-micron calcium silicate and/or Silicon Dioxide can be added to aid in flow through the sachet machine as well as aid in quality and consistency of the final product. The blend is then put through the sachet machine and filled to 24 grams of the homogenous blend. The blend is, for example, sealed one packet at a time.

TABLE 7
is a certificate of analysis table of UreaAide Natural Sparkling Orange.

| Sample Name: | UreaAide Natural | Eurofins | 14167968 |
|---|---|---|---|
| | Sparkling Orange | Sample: | |
| Sample Pass/Fail | PASS | Base Date | 4 Jun. 2024 |
| Project ID | KIDNEYAIDE- | Receipt | Ambient |
| | 20240483 | Condition | Temperature |
| PO Number | NA | Pull Date | 4 Jun. 2024 |
| Lot Number | | Date Started | 4 Jun. 2024 |
| Sample | 24 g | Sampled | Sample |
| Serving Size | | | results apply |
| | | | as received |
| Description | Version 2.0 | Number | 3 |
| | California | Composited | |
| | | Timepoint | Initial |
| Analysis | Result | Specifications | Pass/Fail |
| Package | Commercial foil | | |
| Appearance | pouch | | |
| Characteristic | Sweet and tart | | |
| Odor | odor | | |
| | characteristic of | | |
| | citrus | | |
| Caloric | | | |
| Calculations | | | |
| Calories from Fat | <1.00 Cal/100 g | | |
| Calories from Fat | | | |
| Total | <0.02 g/Serving | | |
| Carbohydrate | Size | | |
| Note | Protein, | | |
| | Carbohydrate | | |
| | and | | |
| | Calories are | | |
| | normally | | |
| | calculated | | |
| | in foods | | |
| | following | | |
| | standard | | |
| | factors. Urea is | | |
| | nitrogen | | |
| | containing but is | | |
| | not a protein | | |
| | nor metabolized | | |
| | for any caloric | | |
| | content. | | |
| Note | The protein, | | |
| | carbohydrate | | |
| | and | | |
| | calories in this | | |
| | sample analysis | | |
| | excludes any | | |
| | contribution | | |
| | from the % | | |
| | measured as | | |
| | urea | | |
| Fatty Acids | | | |
| Calculated as | | | |
| Triglycerides | | | |
| Saturated Fatty | <0.002 | | |
| Acids (Acid Form) | g/Serving Size | | |
| Total Cis | <0.002 | | |
| Unsaturated Fatty | g/Serving Size | | |
| Acids (Acid Form) | | | |
| Monounsaturated | <0.002 | | |
| Fatty Acids (Acid | g/Serving Size | | |
| Form) | | | |
| Polyunsaturated | <0.002 | | |
| Fatty Acids (Acid | g/Serving Size | | |
| Form) | | | |
| | <0.002 | | |
| | g/Serving Size | | |
| Trans Fatty Acids | <0.002 | | |
| (Acid Form) | g/Serving Size | | |
| Omega 3 Fatty | <0.007% | | |
| Acids | | | |
| Fatty Acids | | | |
| Calculated as | | | |
| Triglycerides | | | |
| Omega 6 | 0.007% | | |
| Fatty Acids | | | |
| Omega 7 | <0.007% | | |
| Fatty Acids | | | |
| Omega 9 | <0.007% | | |
| Fatty Acids | | | |
| Total Fatty Acids | 0.002 g/Serving | | |
| | Size | | |
| 4:0 Butyric | <0.007% | | |
| 6:0 Caproic | <0.007% | | |
| 8:0 Caprylic | <0.007% | | |
| 10:0 Capric | <0.007% | | |
| 12:0 Lauric | <0.007% | | |
| 14:0 Myristic | <0.007% | | |
| 14:1 Myristoleic | <0.007% | | |
| 15:0 Pentadecanoic | <0.007% | | |
| 15:1 Pentadecenoic | <0.007% | | |
| 16:0 Palmitic | <0.007% | | |
| 16:1 Palmitoleic | <0.007% | | |
| 17:0 Heptadecanoic | <0.007% | | |
| 17:1 Heptadecenoic | <0.007% | | |
| 18:0 Stearic | <0.007% | | |
| 9c 18:1 Oleic | <0.007% | | |
| 18:2 Linoleic | 0.007% | | |
| 18:3 Gamma | <0.007% | | |
| Linolenic | | | |
| 18:3 Alpha | <0.007% | | |
| Linolenic | | | |
| 18:4 | <0.007% | | |
| Octadecatetraenoic | | | |
| 20:0 Arachidic | <0.007% | | |
| 20:1 Eicosenoic | <0.007% | | |
| 20:2 Eicosadienoic | <0.007% | | |
| 20:3 Eicosatrienoic | <0.007% | | |
| (n3) | | | |
| 20:3 Homogamma | <0.007% | | |
| Linolenic (n6) | | | |
| 20:4 Arachidonic | <0.007% | | |
| (n3) | | | |
| 20:4 Arachidonic | <0.007% | | |
| (n6) | | | |
| 20:5 | <0.007% | | |
| Eicosapentaenoic | | | |
| 21:5 | <0.007% | | |
| Heneicosa- | | | |
| pentaenoic | | | |
| 22:0 Behenic | <0.007% | | |
| 22:1 Erucic | <0.007% | | |
| 22:2 Docosadienoic | <0.007% | | |
| 22:3 Docosatrienoic | <0.007% | | |
| 22:4 | <0.007% | | |
| Docosatetraenoic | | | |
| 22:5 | <0.007% | | |
| Docosapentaenoic | | | |
| (n3) | | | |
| 22:5 | <0.007% | | |
| Docosapentaenoic | | | |
| (n6) | | | |
| 22:6 | <0.007% | | |
| Docosahexaenoic | | | |
| 24:0 Lignoceric | <0.007% | | |
| 24:1 Nervonic | <0.007% | | |
| Total 18:1 trans | <0.007% | | |
| Total 18:1 cis | <0.007% | | |
| Total 18:2 trans | <0.007% | | |
| Total 18:3 trans | <0.007% | | |
| Cholesterol | | | |
| Cholesterol | <0.010 mg/g | | |
| Total Dietary Fiber | | | |
| Total Dietary Fiber | 0.508 g/Serving | | |
| | Size | | |
| Sugar Profile | | | |
| By Ion | | | |
| Chromatography | | | |
| Fructose | 4.36 g/Serving | | |
| | Size | | |
| Sugar Profile | | | |
| By Ion | | | |
| Chromatography | | | |
| Galactose | <0.02 g/Serving | Galactose | Galactose |
| | Size | <0.02 | <0.02 |
| | | g/Serving Size | g/Serving |
| | | | Size |
| Glucose | <0.02 g/Serving | Glucose <0.02 | Glucose |
| | Size | g/Serving Size | <0.02 |
| | | | g/Serving |
| | | | Size |
| Sucrose | <0.02 g/Serving | Sucrose <0.02 | Sucrose |
| | Size | g/Serving Size | <0.02 |
| | | | g/Serving |
| | | | Size |
| Lactose | <0.02 g/Serving | Lactose <0.02 | Lactose |
| | Size | g/Serving Size | <0.02 |
| | | | g/Serving |
| | | | Size |
| Isomaltulose | <0.02 g/Serving | Isomaltulose | Isomaltulose |
| | Size | <0.02 | <0.02 |
| | | g/Serving Size | g/Serving |
| | | | Size |
| Maltose | 0.05 g/Serving | Maltose 0.05 | Maltose 0.05 |
| | Size | g/Serving Size | g/Serving |
| | | | Size |
| Total Sugar | 4.41 g/Serving | Total Sugar | Total Sugar |
| | Size | 4.41 g/Serving | 4.41 |
| | | Size | g/Serving |
| | | | Size |
| Nitrogen, Dumas | | Nitrogen, | Nitrogen, |
| Method | | Dumas | Dumas |
| | | Method | Method |
| Nitrogen | 29.8% | Nitrogen | Nitrogen |
| | | 29.8% | 29.8% |
| Elements by ICP | Elements by ICP | Elements by | Elements by |
| Emission | Emission | ICP Emission | ICP Emission |
| Spectrometry | Spectrometry | Spectrometry | Spectrometr |
| (ICP-OES) | (ICP-OES) | (ICP-OES) | y (ICP-OES) |
| Calcium | 228 ppm | Calcium 228 | Calcium 228 |
| | | ppm | ppm |
| Iron | <2.50 ppm | Iron <2.50 | Iron <2.50 |
| | | ppm | ppm |
| Potassium | 4670 ppm | Potassium | Potassium |
| | | 4670 ppm | 4670 ppm |
| Sodium | 3550 ppm | Sodium 3550 | Sodium 3550 |
| | | ppm | ppm |
| Ash | | Ash | Ash |
| Ash | 1.66% | Ash 1.66% | Ash 1.66% |
| Moisture by | | Moisture by | Moisture by |
| M100_T100 | | M100_T100 | M100_T100 |
| Moisture | 16.5% | Moisture | Moisture |
| | | 16.5% | 16.5% |
| Water Activity | | Water Activity | Water |
| | | | Activity |
| Water Activity | 0.244 | <=0.6 Pass | Pass |
| Temperature | 25.0° C. | | |
| Coagulase Positive | Coagulase | Coagulase | Coagulase |
| Staph. * | Positive Staph. * | Positive | Positive |
| | | Staph. * | Staph. * |
| Staphylococcus | <10 CFU/g | <=10 CFU/g | Pass |
| aureus | | | |
| Salmonella (BAX) | | | |
| PCR Detection | | | |
| Salmonella | Negative/25 g | | |
| Yeast and Mold | | | |
| Plate Count * | | | |
| Yeast Plate | <10 CFU/g | <=200 CFU/g | Pass |
| Mold Plate Count | <10 CFU/g | <=200 CFU/g | Pass |
| Aerobic Plate | | | |
| Count * | | | |
| Standard Plate | 700 CFU/g | <=2000 | Pass |
| Count | | CFU/g | |
| E. coli and | | | |
| Coliforms | | | |
| (Petrifilm) * | | | |
| Total coliforms | <10 CFU/g | | |
| Escherichia Coli | <10 CFU/g | | |
| Urea-DJUPN | | | |
| testing | | | |
| performed in | | | |
| E_DK_VITAM | | | |
| Urea | 15.6 g/Serving | 13.5-16.5 | Pass |
| | Size | g/Serving Size | |
| Method | | | Testing |
| References | | | Location |
| Aerobic | | | EML New |
| Plate Count | | | Berlin |
| (APC) | | | 2345 S 170th |
| | | | St New |
| | | | Berlin, WI |
| | | | 53151 USA |
| FDA BAM Ch. 3 | FDA BAM Ch. 3 | FDA BAM | FDA BAM |
| | | Ch. 3 | Ch. 3 |
| AOAC 966.23 | AOAC 966.23 | AOAC 966.23 | AOAC |
| | | | 966.23 |
| CMMEF Ch. 8 | CMMEF Ch. 8 | CMMEF Ch. 8 | CMMEF Ch. |
| | | | 8 |
| Ash (ASHM_S) | | | Food |
| | | | Integrity |
| | | | Innovation- |
| | | | Madison |
| | | | 6304 Ronald |
| | | | Reagan Ave |
| | | | Madison, WI |
| | | | 53704 USA |
| Official | | | |
| Methods of | | | |
| Analysis | | | |
| of AOAC | | | |
| INTER- | | | |
| NATIONAL, | | | |
| 18th Ed., Method | | | |
| 923.03, AOAC | | | |
| INTER- | | | |
| NATIONAL, | | | |
| Gaithersburg, | | | |
| MD, | | | |
| USA, (2005). | | | |
| (Modified) | | | |
| Caloric | | | Food |
| Calculations | | | Integrity |
| (CALC_S) | | | Innovation- |
| | | | Madison |
| | | | 6304 Ronald |
| | | | Reagan Ave |
| | | | Madison, WI |
| | | | 53704 USA |
| United States | | | |
| Department of | | | |
| Agriculture, | | | |
| “Energy | | | |
| Value of Foods,” | | | |
| Agriculture | | | |
| Handbook No. 74, | | | |
| pp 2-11 (1973). | | | |
| Code of Federal | | | |
| Regulation, | | | |
| Title 21, | | | |
| Part 101.9, pp. | | | |
| 24-25 | | | |
| Method | | | Testing |
| References | | | Location |
| Cholesterol | | | Food |
| (CHOLSTRL_S) | | | Integrity |
| | | | Innovation- |
| | | | Madison |
| | | | 6304 Ronald |
| | | | Reagan Ave |
| | | | Madison, WI |
| | | | 53704 USA |
| | AOCS Official | | |
| | Method Ce 12- | | |
| | 16, Sterols and | | |
| | Stanols in Foods | | |
| | and Dietary | | |
| | Supplements | | |
| | Containing | | |
| | Added | | |
| | Phytosterols | | |
| | (Modified). | | |
| Coagulase | Bennett, R. W., | | EML New |
| Positive | and Lancette, G. | | Berlin |
| Staph. | A., “Chapter 12- | | 2345 S 170th |
| (STAPHPC) | Staphylococcus | | St New |
| | aureus,” | | Berlin, WI |
| | Bacteriological | | 53151 USA |
| | Analytical | | |
| | Manual, Eighth | | |
| | Ed., Revision A, | | |
| | U. S. Food and | | |
| | Drug | | |
| | Administration: | | |
| | Silver Spring, | | |
| | MD (1998), | | |
| | Revised Janunary | | |
| | 2001. | | |
| | (Modified). | | |
| E. coli and | AOAC 991.14 | | EML New |
| Coliforms | | | Berlin |
| (Petrifilm) | | | 2345 S 170th |
| (ECCOLIPET) | | | St New |
| | | | Berlin, WI |
| | | | 53151 USA |
| Elements by ICP | Official Methods | | Food |
| Emission | of Analysis of | | Integrity |
| Spectrometry | AOAC | | Innovation- |
| (ICP- | INTER- | | Madison |
| OES) (ICP_S) | NATIONAL, | | 6304 Ronald |
| | Method | | Reagan Ave |
| | 984.27, 985.01, | | Madison, WI |
| | and 2011.14, | | 53704 USA |
| | AOAC | | |
| | INTER- | | |
| | NATIONAL, | | |
| | Gaithersburg, | | |
| | MD, USA. | | |
| | (Modified) | | |
| Fatty Acids | Official Method | Official | Food |
| Calculated as | No. 996.06, | Methods and | Integrity |
| Triglycerides | Official Methods | Recommended | Innovation- |
| (FALT_S) | of Analysis of | Practices of | Madison |
| | the AOAC | the AOCS, | 6304 Ronald |
| | INTER- | Official | Reagan Ave |
| | NATIONAL | methods Ce | Madison, WI |
| | (modified), 19th | 2b-11 (2011), | 53704 USA |
| | Ed., AOAC | Ce 1h-05 | |
| | INTER- | (2009), Ce | |
| | NATIONAL: | 1j-07 (2013), | |
| | Gaithersburg, | Ce 2-66 | |
| | Maryland | (2009), The | |
| | (2012). | American Oil | |
| | | Chemists′ | |
| | | Society, | |
| | | Champaign, IL | |
| | | (modified). | |
| Moisture by | Official Methods | | Food |
| M100_T100 | of Analysis of | | Integrity |
| (M100T100_S) | AOAC | | Innovation- |
| | INTER- | | Madison |
| | NATIONAL, | | 6304 Ronald |
| | 18th Ed., | | Reagan Ave |
| | Methods 925.09 | | Madison, WI |
| | and 926.08, | | 53704 USA |
| | AOAC | | |
| | INTER- | | |
| | NATIONAL, | | |
| | Gaithersburg, | | |
| | MD, | | |
| | USA, (2005). | | |
| | (Modified). | | |
| Nitrogen, Dumas | Official Methods | | Food |
| Method | of Analysis of | | Integrity |
| (DGEN_S) | AOAC | | Innovation- |
| | INTER- | | Madison |
| | NATIONAL, | | |
| | 18th Ed., | | 6304 Ronald |
| | Methods 968.06 | | Reagan Ave |
| | and 992.15, | | Madison, WI |
| | AOAC | | 53704 USA |
| | INTER- | | |
| | NATIONAL, | | |
| | Gaithersburg, | | |
| | MD, USA, | | |
| | (2005). | | |
| | (Modified) | | |
| Organoleptic | INTERNAL SOP | | Food |
| Evaluation | DESIGNED TO | | Integrity |
| (ORGE) | SUPPLEMENT | | Innovation- |
| | THE UNITED | | Madison |
| | STATES | | 6304 Ronald |
| | PHARMACOPEIA, | | Reagan Ave |
| | 25TH EDITION, | | Madison, WI |
| | PAGE 2363, USP | | 53704 USA |
| | CONVENTION, | | |
| | INC., | | |
| | ROCKVILLE, | | |
| | MD., 2002. | | |
| Project Setup Fee | AOAC 2003.09; | | Food |
| (STABILITY) | DuPont | | Integrity |
| | Qualicon BAX | | Innovation- |
| | System User | | Madison |
| | Guide, Part | | 6304 Ronald |
| | Number 2CQ- | | Reagan Ave |
| | 049. 13-0713- | | Madison, WI |
| | V3.3, 2005- | | 53704 USA |
| | 2013. | | |
| Salmonella (BAX) | AOAC 2003.09; | | EML New |
| PCR Detection | DuPont | | Berlin |
| (SALBAX) | Qualicon BAX | | 2345 S 170th |
| | System User | | St New |
| | Guide, Part | | Berlin, WI |
| | Number 2CQ- | | 53151 USA |
| | 049. 13-0713- | | |
| | V3.3, 2005- | | |
| | 2013. | | |
| Sugar Profile | AOAC 2018.16 | | Food |
| By Ion | Sugar Profile in | | Integrity |
| Chromatography | Food, Dietary | | Innovation- |
| (SGIC_2_S) | Supplements, | | Madison |
| | Pet Food and | | 6304 Ronald |
| | Animal Feeds | | Reagan Ave |
| | (modified) | | Madison, WI |
| | | | 53704 USA |
| Total Dietary | Official Methods | | |
| Fiber | of Analysis of | | |
| (TDFL_S) | AOAC | | |
| | INTER- | | |
| | NATIONAL | | |
| | 18th Ed., | | |
| | Method 991.43, | | |
| | AOAC | | |
| | INTER- | | |
| | NATIONAL, | | |
| | Gaithersburg, | | |
| | MD, | | |
| | USA, (2005). | | |
| | (Modified) | | |
| Urea-DJUPN | Test performed | | External |
| testing | by a third party | | |
| performed in | laboratory | | |
| E_DK VITAM | | | |
| (MISC_SEND) | Official Methods | | Food |
| Water Activity | of Analysis of | | Integrity |
| (WACT_S) | AOAC | | Innovation- |
| | INTER- | | Madison |
| | NATIONAL, | | 6304 Ronald |
| | Method | | Reagan Ave |
| | 978.18, AOAC | | Madison, WI |
| | INTER- | | 53704 USA |
| | NATIONAL, | | |
| | Gaithersburg, | | |
| | MD, | | |
| | USA (Modified). | | |
| Yeast and Mold | FDA BAM | | EML New |
| Plate Count | Chapter 18 | | Berlin |
| (YM_SPRD) | (modified) | | 2345 S 170th |
| | | | St New |
| | | | Berlin, WI |
| | | | 53151 USA |

Testing Location(s) Released on Behalf of Eurofins by Edward Ladwig-President Eurofins Food
Chemistry Testing Madison Food Integrity Innovation-Madison
Eurofins Food Chemistry Testing Madison, Inc.
6304 Ronald Reagan Ave Madison WI 53704 800-675-8375 2918.01

What is claimed is:

1. A powder formulation comprising:
(a) an amount of sparkling urea effective for treatment of hyponatremia;
(b) calcium silicate, the calcium silicate having a particle size in the range of 3 to 8 microns, wherein less than 10% of the particles by weight have a diameter less than 3 microns or greater than 8 microns.

2. The composition of claim 1, wherein the urea comprises 63.16% of the formulation weight.

3. The composition of claim 1, wherein the urea is a sparkling UreaAide formulation.

4. The composition of claim 1, further comprising a flavoring agent.

5. The composition of claim 1, wherein the calcium silicate has a particle size of 8 microns.

6. The composition of claim 1, wherein the sparkling urea has a dose of 15 grams.

7. The composition of claim 1, wherein the sparkling urea is mixed with a liquid.

8. The composition of claim 1, wherein the sparkling urea is mixed with food.

9. A powder formulation comprising:
(a) an amount of sparkling urea effective for treatment of hyponatremia;
(b) calcium silicate, the calcium silicate having a particle size of 8 microns.

10. The powder formulation of claim 9, further comprising a flavoring agent.

11. The powder formulation of claim 9, wherein the urea comprises 63.16% of the formulation weight.

12. The powder formulation of claim 9, wherein the urea is a sparkling UreaAide formulation.

13. The powder formulation of claim 9, wherein the sparkling urea has a dose of 15 grams.

14. The powder formulation of claim 9, wherein the sparkling urea is mixed with a liquid.

15. The powder formulation of claim 9, wherein the sparkling urea is mixed with food.

16. A method of treating a subject with hyponatremia comprising:
(a) administering sparkling urea effective for treatment of hyponatremia;
(b) administering calcium silicate, the calcium silicate having a particle size in the range of 3 to 8 microns, wherein less than 10% of the particles by weight have a diameter less than 3 microns or greater than 8 microns,
wherein the treatment is administered in single-use urea packet.

17. The method of claim 16, wherein the sparkling urea has a dose of 15 grams.

18. The method of claim 1, wherein the sparkling urea formulation comprises a bitter blocker.

19. The method of claim 1, wherein the urea is a sparkling UreaAide formulation.

20. The composition of claim 16, further comprising a flavoring agent.
